(19) United States
(12) Patent Application Publication
(10) Pub. No.: US 2025/0174320 A1
(43) Pub. Date: May 29, 2025

(54) Method And System For Processing Medical Data

(71) Applicants: Ryan Grant Renne, Atlanta, GA (US); Nathaniel James Dieckhaus, Atlanta, GA (US)

(72) Inventors: Ryan Grant Renne, Atlanta, GA (US); Nathaniel James Dieckhaus, Atlanta, GA (US)

(21) Appl. No.: 18/522,022

(22) Filed: Nov. 28, 2023

(51) Int. Cl. G16H 10/60; A61N 5/10; G10L 15/183; G10L 15/22; G16H 20/40; G16H 50/20

(52) U.S. Cl. CPC G16H 10/60; A61N 5/103; G10L 15/183; G10L 15/22; G16H 20/40; G10L 2015/223; G16H 50/20

(57) ABSTRACT

A method for processing medical data by a client application and a chatbot, includes: receiving, by the client application, a user command comprising natural language, the user command further comprising personal information; pre-processing, by the client application, the user command to mask the personal information; invoking the chatbot to determine an action and/or an argument from the pre-processed user command; and processing the medical data, by the client application, using a feature of the client application to perform the chatbot-determined action and/or argument on the medical data.

### FIELD

Embodiments herein relate to methods and systems for processing medical data. In particular, the methods and systems are for processing medical data associated with radiotherapy.

### BACKGROUND

Large amounts of medical data are created in hospitals. The medical data may be stored electronically. In the field of radiotherapy, medical data may include any one or more of patient data (e.g., an electronic medical record (EMR) or electronic health record (EHR)), medical imaging data, treatment plan information, dose-volume histograms (DVHs), dose information, dosimetric metrics, clinical outcomes, instruction for use (IFU) and others. By centralizing such medical data in a repository (such as Elekta Proknow®), the data may be accessed and processed to derive new insights, enable peer review, improve adherence to standard protocols, and improve the quality of radiotherapy treatment plans.

Radiotherapy or radiation therapy can be described as the use of ionizing radiation to damage or destroy unhealthy cells in both humans and animals. Unhealthy cells may include cancerous cells, for example. The ionizing radiation may be directed to tumors on the surface of the skin or deep inside the body. Common forms of ionizing radiation include X-rays and charged particles. An example of a radiotherapy technique is Gamma Knife® or Leksell Gamma Knife® where a patient is irradiated using a number of lower-intensity gamma rays that converge with higher intensity and high precision at a targeted region (e.g., a tumor). Another example of radiotherapy comprises using a linear accelerator (“linac”), whereby a targeted region is irradiated by high-energy particles (e.g., electrons, high-energy photons, and the like). In another example, radiotherapy is provided using a heavy charged particle accelerator (e.g., protons, carbon ions, and the like).

Medical imaging data for radiotherapy comprises CT images, MR images and/or other image modalities. Patient data may include personal information (e.g., personally identifiable information (PII) and/or protected health information (PHI)). PII comprises any information that can be directly or indirectly linked to an individual's identity (e.g. identification numbers, passport numbers, email address, photo, biometric information or any other information). PHI may comprise information about health status, provision of health care, or payment for health care, that can be linked to a specific individual (e.g., Names, geographical information, dates, phone/fax numbers, email address, social security number, medical record number, health insurance beneficiary number, account numbers, certificate/license numbers, vehicle identifiers, device identifiers, URLs, IP address, biometric identifiers, full face photographs, or any other unique identifying feature).

Medical imaging data and or patient data may be stored using the Digital Imaging and Communications in Medicine (DICOM) standard. Medical imaging data may also include anatomical structures (e.g., planned target volume, target, organ(s) at risk, etc.) obtained from a contouring procedure. Further, dose at the different structures may be included. Treatment plan information may include details of the treatment delivery (e.g., the number of beams, the machine, the modality, the beam energy, and the machine output in monitor units (MU)).

Users may process medical data to, for example, perform peer review, train, and derive insights. This may lead to improved radiotherapy treatment plans and improved clinical outcomes.

There is a need for improved methods and systems for processing medical data where users may process medical data more easily, while protecting patient data.

### BRIEF DESCRIPTION OF DRAWINGS

Systems and methods in accordance with non-limiting examples will now be described with reference to the accompanying figures in which:

FIG. 1 is a flowchart of a method for processing medical data by a client application and a chatbot;

FIG. 2 is a schematic illustration of a system for processing medical data using a client application and a chatbot;

FIG. 3 is an example of processing medical data;

FIG. 4 is a flowchart of a method for processing medical data by a client application and a chatbot;

FIG. 5 is a schematic illustration of a language model used in a chatbot; and,

FIG. 6 is a block diagram of an apparatus, device, system or machine according to various embodiments.

### DETAILED DESCRIPTION

As described above, the processing of medical data may enable the deriving of new insights, peer review, improved adherence to standard protocols, and improved quality of radiotherapy treatment planning. Different users or clinical centers may use different protocols or recipes to arrive at suitable treatment plans. Further, the devised treatment plans may be dependent on the experience of the healthcare professional. Thus, treatment plans targeting the same conditions may vary across users and centers. Processing of treatment plans and their associated outcomes may provide new insights and lead to improved treatment planning.

The following describes methods and systems that improve the processing of medical data by a user. Medical data may relate to real-world information. In particular, the devised methods and systems facilitate the processing of medical data by allowing the user to enter a user query (also referred to as a command) in natural language using a client application and a chatbot. A chatbot is a type of software agent configured to converse with a user. A chatbot may also be referred to as a conversational agent or system, a smart assistant, or an artificial intelligence (AI) agent. Natural language refers to language that that occurs naturally in human communication. For example, a user (e.g., a treatment planner, dosimetrist, clinician, health care worker, or analyst) would converse with a human colleague using natural language. Natural language can be spoken or written. Natural language is different from a structured language such as a computer programming language. A client application is a user facing computer program that may access data sources and connect to other applications and functions. For example, the client application may access and/or connect to a chatbot. The client application is a computer program configured to process medical data. In particular, the client application may be permitted to process patient data. For example, the client application may include physical, electronic, and procedural safeguards that limit access to authorized users. Authorized users are users that are permitted to access patient data. Further, any transmission to and from the client application may be encrypted.

By using natural language (instead of, e.g., a computer programming language) the ease of use is increased. Together with easier processing of medical data, the devised methods and systems preserve the privacy of personal information included in the user query. The methods and systems herein address a technical problem arising in the field of processing medical data (in particular, radiotherapy medical data), namely, how to facilitate the processing of medical data, by receiving commands from a user in natural language, while meeting privacy requirements.

According to a first aspect, there is provided a method for processing medical data by a client application and a chatbot, the method comprising: receiving, by the client application, a user command comprising natural language, the user command further comprising personal information; pre-processing, by the client application, the user command to mask personal information; invoking the chatbot to determine (or, directly determining, by control of the chatbot), an action and/or an argument from the pre-processed user command; and processing the medical data, by the client application, using a feature of the client application to perform the chatbot-determined action and/or the determined argument on the medical data.

The user command is expressed in natural language. The user command may comprise an action and/or an argument. E.g., the user command may comprise an action only, an argument only, or an action and an argument. Examples of actions are instructions to ‘create’, ‘copy’, ‘delete’, ‘retrieve’, ‘modify’, ‘combine’, ‘search’ amongst others. An argument refers to an object (or item) to which, or based on which, an action is applied. For example, an argument may be an attribute of medical data, such as a treatment record, a treatment plan, a collection of data structures, a patient attribute, etc. An example of a user command is: “Copy treatment plan for John Doe into a collection called ‘treatments for prostate’”. Here, the action is ‘copy’ and the arguments would be ‘collection’, ‘treatment plan’, ‘John Doe’, ‘treatments for prostate’.

The chatbot is configured to process commands in natural language and is used to analyze the command (also referred to as a user query) provided by the user. This enables the user to formulate commands using natural language rather than being restricted to a particular syntax.

The chatbot may be separate from the client application. For example, the chatbot may be operated by a third party. The chatbot may not be permitted to process and/or receive personal information associated with the medical data. On the other hand, the client application is configured to process medical data (e.g., it may be permitted to access and/or process medical data that comprises personal information). Personal information comprises information that may be linked to an individual, as described herein. The method combines the use of the chatbot to analyze user instructions in natural language together with the use of the client application to process medical data containing personal information (that may only be processed by the client application). Thus, the accuracy and ease of use of the system is improved (since a user may interact with the system using natural language rather than having to use a specific syntax) while meeting any privacy requirements (which are met by the client application, but which may not be met by the chatbot).

This is achieved as follows. The client application receives a user command, the user command comprising natural language. The user command is pre-processed by the client application to mask personal information that may be present. Masking may also be referred to as anonymizing. The pre-processed user command, which does not comprise personal information, is then presented to the chatbot. The chatbot is then invoked (e.g., with a prompt, a call, a request, or another instruction) to determine an action and/or an argument from the pre-processed user command. The chatbot does not see the personal information (since this has been masked by the client application). The client application then processes the medical data, using the action and/or the argument determined by the chatbot.

The client application may then output an indication that the medical data has been processed to the user or may output the processed medical data.

In an embodiment, the client application determines whether the processed medical data comprises personal information. In response to determining that personal information is present, the processed medical data is masked by the client application. An indication that the medical data has been processed is generated by the chatbot using the masked medical data. The generated indication is output by the client application. Thus, any personal information that may be present in the processed medical data is not made available to the chatbot (as the chatbot only sees the masked medical data). The natural language capabilities of the chatbot may still be used to generate an indication. Such an indication may be more easily understood by a user.

In an embodiment, the client application determines whether the user command comprises personal information, and in response to personal information being present, the client application pre-processes the user command to mask the personal information.

In an embodiment, the chatbot is configured to: determine an action from the pre-processed command by comparing the pre-processed command to a set of reference actions. In an embodiment, the chatbot is configured to: determine an argument from the pre-processed command by comparing the pre-processed command to a set of reference arguments. By comparing to reference actions/arguments, the determination may be more accurate.

In an embodiment, the chatbot-determined action comprises any one or more of: searching for patient records, querying patient records, creating a collection of patient records, modifying patient records, retrieving medical images, or searching a data source for an item. These actions may be performed on the medical data using an appropriate feature that is provided by the client application or by another computer-implemented software program, library, or component.

In an embodiment, in response to the chatbot-determined action comprising searching a data source for an item, the chatbot determines an embedding that corresponds to the item to be searched, and the client application compares the determined embedding with a set of reference embeddings to obtain a candidate embedding. The candidate embedding is an embedding selected from the set of reference embeddings. The candidate embedding represents the reference embedding that is similar to the item being searched. The client application retrieves information that corresponds to the candidate embedding from the data source. The accuracy and speed of the search may be improved.

In an embodiment, masking personal information comprises identifying personal information in the user query and then either removing the personal information, or replacing the personal information by an anonymized text string. This is performed by the client application. Thus, personal information is not available to the chatbot.

The personal information that is to be masked (masked personal information) may be stored at the client application. Thus, only the client application may see the personal information.

In an embodiment, the client application uses the stored personal information, together with the chatbot-determined action and/or argument to process the medical data using a feature of the client application. Thus, the processing of the medical data based on personal information may be carried out.

In an embodiment, when masking comprises replacing the personal information by an anonymized text string, the client application replaces the anonymized text string by the stored personal information and then processes the medical data. Thus, the processing of the medical data based on personal information may be carried out by the feature of the client application.

The chatbot may comprise a language model. The chatbot may comprise a generative pre-trained transformer (GPT).

Medical data may comprise any one or more of electronic medical records, electronic health records, radiotherapy plans, patient metrics, and medical imaging data.

According to a second aspect, there is provided a system for processing medical data by a client application and a chatbot, the system comprising: processing circuitry; and memory, including instructions stored thereon, which, when executed by the processing circuitry, causes the processing circuitry to perform any of the above methods.

According to a third aspect, there is provided a non-transitory computer-readable medium with instructions stored thereon that, when executed by a processor of a computing device, cause the processor to perform any of the above methods.

According to another example, there is provided a computer program comprising instructions which, when the program is executed by a computer, cause the computer to carry out any of the above methods.

A computer program and/or the code for performing such methods may be provided to a system (such as the system according to the second aspect) on one or more computer readable media or, more generally, a computer program product.

The methods are computer-implemented methods. Since some methods in accordance with examples can be implemented by software, some examples encompass computer code provided to a general purpose computer on any suitable carrier medium. The carrier medium can comprise any storage medium such as a floppy disk, a CD ROM, a magnetic device or a programmable memory device, or any transient medium such as any signal e.g. an electrical, optical or microwave signal. The carrier medium may comprise a non-transitory computer readable storage medium.

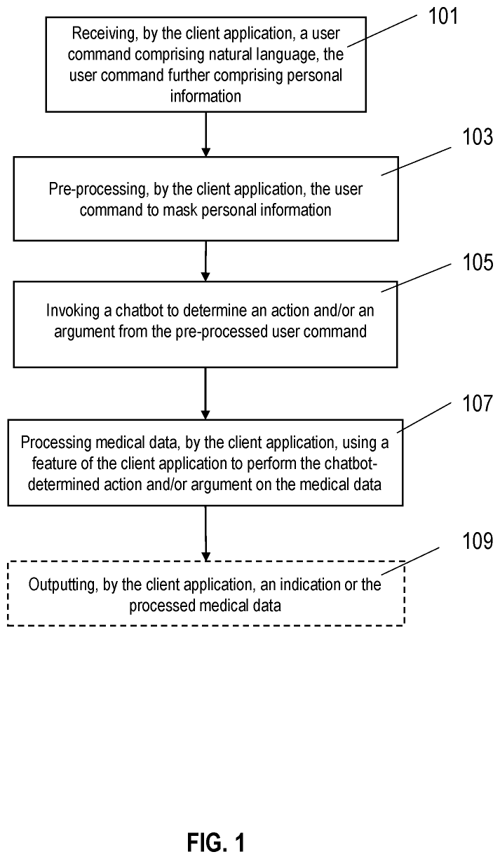

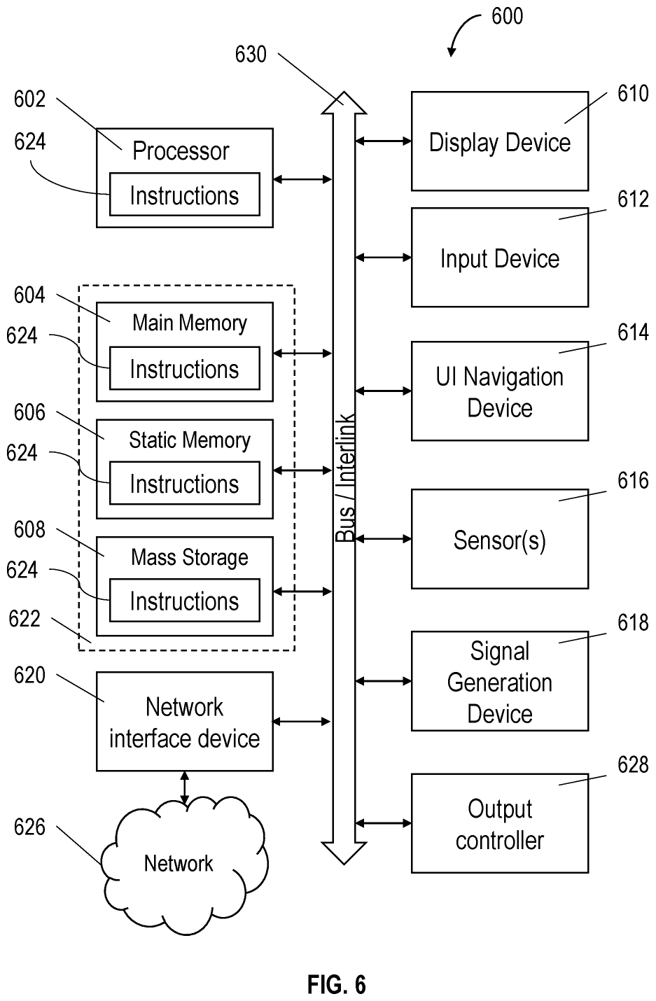

FIG. 1 shows a flowchart of a method for processing medical data by a client application and a chatbot. The method is for processing medical data associated to radiotherapy. The method of FIG. 1 may be performed by the system 600 described in relation to FIG. 6.

At operation 101, the client application receives a user command, the user command comprising natural language.

The user command may be provided by a user in the form of a speech signal or in the form of a text string. When the command is in the form of a speech signal, the speech is transcribed into a text string using an automatic speech recognition (ASR) tool. The user command may be obtained using any of the input means described in relation to FIG. 6

The client application is a computer program configured to process medical data via one or more features (e.g., functions, methods, services, or components provided or controlled by the client application). The client application is configured to access and analyze medical data such as any one or more of patient data, medical imaging data, treatment plan information, dose-volume histograms (DVHs), dose information, dosimetric metrics, and clinical outcomes. The client application may be permitted to process patient data. For example, the client application may include physical, electronic, and procedural safeguards that limit access to authorized users. Authorized users are users that are permitted to access patient data. Further, any transmission to and from the client application may be encrypted. Optionally, the client application comprises procedures for anonymizing medical data. For example, the client application may comprise procedures for anonymizing selected attributes in DICOM data prior to allowing access to said data. The anonymization procedure may comprise any one or more of replacing an attribute by a randomly generated value, replacing an attribute by a pre-defined value, or removing an attribute.

At operation 103, the user command provided by the user is pre-processed by the client application. The purpose of the pre-processing is to mask personal information that may be included in the user command. This operation is performed prior to operation 105, which is performed by a chatbot. Operation 103 may also be referred to as anonymization.

Additionally and optionally, pre-processing the user command to mask personal information comprises: (i) identifying personal information in the user command, and (ii) masking said information.

Masking of the personal information may be performed by either replacing the identified information by a pre-defined value or replacing the identified information by a string of random values. The text string comprising the pre-defined value or the string of random values may be referred to as an anonymized text string. Yet optionally, the string of random values may be based on a private key, resulting in an untraceable anonymized text string.

Alternatively, masking the personal information comprises removing the identified information from the user command.

Additionally and optionally, the personal information that is to be masked is stored in a list or a lookup table at the client application for use in a subsequent operation. For example, at operation 107 (described below), the client application may use the stored personal information (optional).

Identifying personal information may be performed as follows:
One or more Regular Expressions (RegEx) may be used to identify features such as dates or other features that follow a specific format. A RegEx is a sequence of characters that specifies a pattern to match in a text string. For example, a RegEx to identify a date may have a format of ‘dd.mm.yyyy’, ‘MON-DD-YYYY’, or any other format used for dates. Similarly, a RegEx may be used to identify features that follow a particular pattern such as social security numbers, addresses, phone/fax numbers, email addresses, etc.
Additionally and optionally, a set of predetermined rules is used to identify words that relate to personal information. An example is gender which is indicated by terms such as ‘male’, ‘female’, ‘he’, ‘she’, ‘they’, etc.
Additionally and optionally, any names in the text string are identified by using a pre-trained classifier. The pre-trained classifier is located at the client application. For example, a user command may be broken down into its constituent words. The pre-trained classifier is configured to analyze a word and determine whether the word is likely to be a name. The likelihood that a word is a name may be represented by a score, for example.

Optionally, the score is between 0 and 1. The score associated with a word may be compared to a predetermined threshold and a word having a score that meets this threshold is considered to represent a name. In an example, the predetermined threshold is 0.7, and any word scoring 0.7 or above is considered to be a name.

Optionally, for a word that is considered to be a name, the word is compared to a database of patient names that are present in the medical data. This is to determine whether the user had entered a known patient name, and if a match is detected the name may be replaced.

The pre-trained classifier may be implemented using a machine learning algorithm. Examples of suitable algorithms include: naïve Bayes classifiers, support vector machines, or deep learning algorithms.

The pre-trained classifier may be trained as follows. The classifier may be trained on a labelled data set comprising 5000 to 10000 text strings containing patient names in various formats, in various languages. An example of a classifier is a Named Entity Recognition (NER).

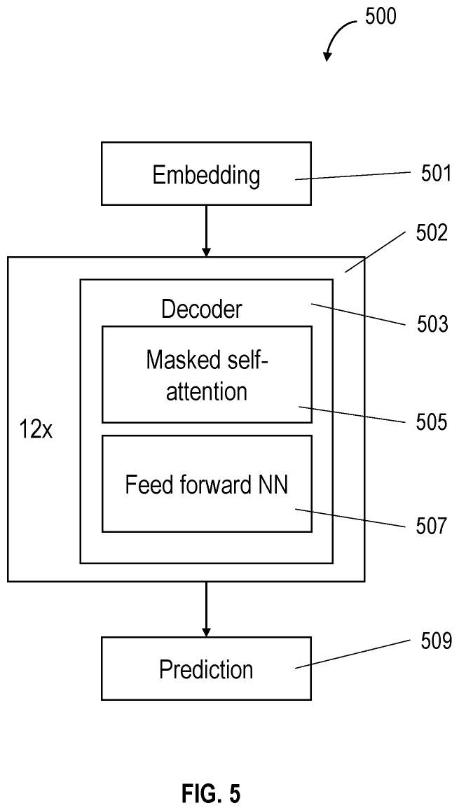

At operation 105, the chatbot is invoked (e.g., with a prompt, a call, a request, or another instruction) by the client application, and the pre-processed user command, provided by the client application at operation 103, is analyzed by the chatbot. The chatbot is configured to determine an action and/or an argument from the pre-processed user command. An action may also be referred to as a function. The chatbot may comprise a trained model. For example, the chatbot comprises a language model (LM). An example of a chatbot is described in relation to FIG. 5.

Unlike the user command, the pre-processed user command does not include personal information. Personal information has been masked by the client application at operation 103. Thus, the chatbot does not ‘see’ personal information. Privacy may be maintained. The pre-processed user command comprises a text string in natural language. The chatbot is configured to identify an action and/or an argument included in the pre-processed user command (which comprises a natural language text string), and return said action and/or argument. The input to the chatbot may be referred to as a prompt, while the output returned by the chatbot may be referred to as a completion.

Additionally and optionally, the chatbot is configured to determine an action and/or argument from the prompt by comparing the pre-processed user command to a set of reference actions and/or arguments. The chatbot may be configured to recognize reference actions and/or arguments at operation 105 as follows. The prompt provided to the chatbot may include a set of reference actions and/or reference arguments, together with the pre-processed user command. Thus, the chatbot may determine an action from the pre-processed user command, based on the pre-processed user command and a set of reference actions. Further, the chatbot may determine an argument form the pre-processed user command, based on the pre-processed user command and a set of reference arguments.

For example, when the user query is “Create a collection of female patients”, the term ‘female’ may be masked (operation 103) such that the pre-processed user query is “Create a collection of XXXX patients”. Here, ‘XXXX’ represents either a predefined string or a string of randomly generated characters that has been used to replace the term ‘female’. An example of a prompt provided to the chatbot may then be:

TABLE 1

```
Pre-processed User query: <<“Create a collection of XXXX
patients”>>
  functions: [{
    “name”: “createCollection”,
    “description”: “Creates a new collection of
patients”,
    “arguments”: [{
      “name”: “collection_name”,
      “description”: “The name to use when creating
the collection”,
      “type”: “string”
  }, {
    “name”: “patient_attribute”,
    “description”: “The patient attribute to use when
    filtering patients to add to the collection”,
    “type”: “string”
    }]
    }, {
    “name”: “copyNotes”,
    “description”: “Copies patient notes from a source
patient to a target patient”,
    “arguments”: [{
      “name”: “source_name”,
      “description”: “The source patient name to copy
notes from”,
      “type”: “string”
    }, {
      “name”: “target_name”,
      “description”: “The target patient name to copy
notes to”,
      “type”: “string”
    }]
}]
```

In the above example, ‘functions’ represent information included in the prompt in addition to the pre-processed user query. The ‘functions’ inform the behavior of the chatbot. The ‘functions’ comprise a list of available functions (reference actions) and include the name, description and reference arguments for each function. In the above example, two functions (reference actions) are provided. One function is to create a collection (‘createCollection’) and arguments (reference arguments) include the name of the collection (‘collection_name’) and a patient attribute used to select which patients to add (‘patient_attribute’). Another function is to copy patient notes (‘copyNotes’) and arguments (reference arguments) include the name of the patient name to copy from (‘source_name’) and the name to the patient to copy to (‘target_name’).

The chatbot identifies, from the pre-processed user query, an action from the available list of functions to execute, and any arguments. The action to execute and any associated arguments may be referred to as an instruction. In this example, the function to call is ‘create’ and the arguments are ‘collection’ and the patient attribute ‘XXXX’. The output returned by the chatbot is then the function corresponding to ‘createCollection’ and the arguments ‘XXXX collection’ and ‘XXXX’. For example, the output comprises the following instruction:

TABLE 2

```
function_call: {
  “name”: “createCollection”,
  “arguments: [
    “XXXX collection”,
    “XXXX”
  ]
}
```

The client application then reads the function name and arguments from the response of the chatbot and calls the indicated function with the suggested arguments, after replacing “XXXX” with ‘female’.

At operation 107, medical data is processed, by the client application, using the action and/or argument determined by the chatbot in operation 105 within one or more features of the client application. As noted above, the one or more features of the client application may include functions, methods, services, or components that are provided or controlled by the client application, including features specially designed for medical data processing. Processing medical data comprises performing, at the client application using the feature, the determined action using the determined argument (when present). The determined action may comprise any of: searching for patient records (e.g. ‘Find the records for patient X’), querying patient records (e.g. ‘Find all records that mention ‘lung’’), creating a new collection that comprises one or more patient records, modifying patient data, retrieving a patient record from patient data, retrieving a medical image, or searching a data source for an item, among other examples.

Additionally and optionally, processing the medical data comprises using the masked personal information. The masked personal information may be stored at the client application as described in relation to operation 103. The anonymized text string or strings may be replaced by stored personal information such that the medical data may be processed by the client application. The replacing of anonymized string or strings by stored personal information may be referred to as unmasking or de-anonymizing.

In relation to the example above where the user query is “Create a collection of female patients”, the pre-processed user query is “Create a collection of XXXX patients”, the determined action is ‘createCollection’, and the determined arguments are ‘XXXX collection’ and ‘XXXX’, and the masked personal information corresponding to ‘XXXX’ is ‘female’ (stored at the client application). At the client application, the anonymized text string ‘XXXX’ is replaced by its corresponding stored personal information ‘female’. The client application processes the medical data to generate a collection of female patients.

At operation 109, either the processed medical data and/or an indication that medical data has been processed is outputted. Outputting the processed medical data comprises any of displaying the processed medical data on a display unit, and storing/transferring the processed medical data to memory. In the example with the user query being “Create a collection of female patients”, the newly created collection may be outputted e.g., by displaying on a display unit, providing a link to the collection, or by creating an instance of a collection and storing it in memory. Alternatively, outputting an indication comprises displaying a visual indication (such as “Collection of female patients created!”) on a display unit or providing an audio indication.

Note that operation 109 is optional. The processed medical data from operation 107 may be stored by the client application.

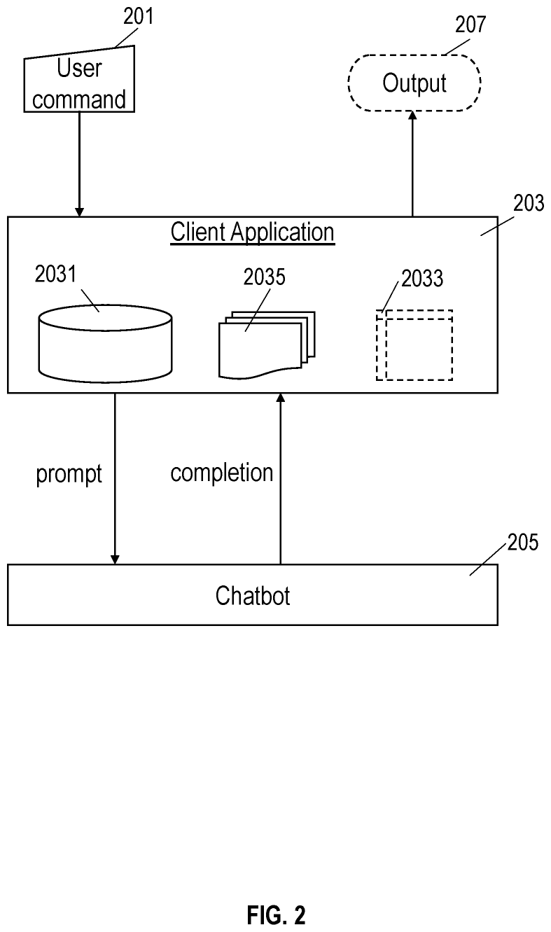

FIG. 2 shows a schematic illustration of a system for processing medical data using a client application and a chatbot. The system may be implemented as described in relation to FIG. 6. The system may implement the method described in relation to FIG. 1.

A user command 201 (also referred to as a user query) is received from a user. The user command 201 is pre-processed by the client application 203 as described in relation to FIG. 1. The client application 203 has access to medical data 2031. The client application 203 also has access to processed medical data 2035. Processed medical data 2035 is generated at the client application 203 in response to receiving an instruction (as described in relation to FIG. 1). Processed medical data 2035 may be stored at the client application 203. An instruction comprises an action to perform and/or an argument. Instructions are obtained from a completion generated by the chatbot 205.

The chatbot 205 receives a prompt from the client application 203. An example of a chatbot is provided in FIG. 5. The prompt comprises a pre-processed user query (pre-processed user command) as described in relation to FIG. 1. Optionally, the prompt comprises a set of reference actions and/or reference arguments, together with the pre-processed user query as described in relation to FIG. 1. The chatbot 205 generates a completion. The completion comprises a determined action and/or a determined argument as described in FIG. 1. The completion corresponds to an instruction that is used by the client application 203 to obtain processed medical data 2035. The processing of medical data is described in relation to FIG. 1.

An output 207 may be provided by the client application 203. The output 207 is as described in relation to FIG. 1.

Additionally and optionally, when personal information in the user command 201 is masked, said personal information is stored in a list or a lookup table 2033 at the client application 203 for use in a subsequent operation (as described in relation to FIG. 1). The client application 203 may have access to said list or lookup table 2033. For example, as described in relation to FIG. 1, the client application 203 may use the personal information in the list 2033 to unmask (or de-anonymize) the identified action and/or argument. The unmasked action and/or argument may then be used to process medical data 2031.

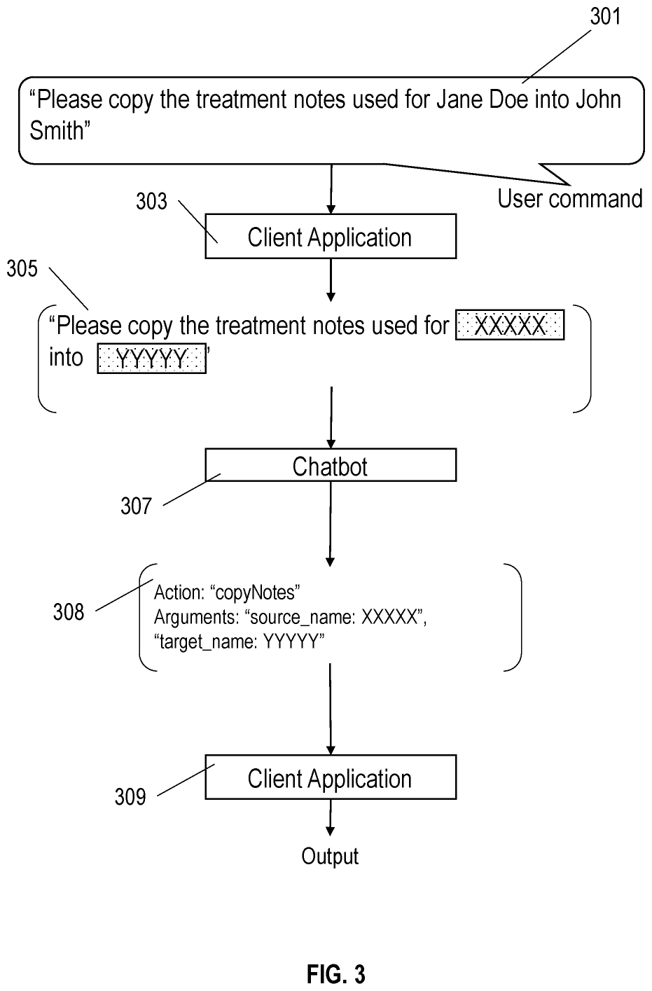

FIG. 3 shows an example of processing medical data using methods and systems described herein. In this example, the following user command 301 is provided: “Please copy the treatment notes used for Jane Doe into John Smith”. As discussed herein, the user command may be provided by a user in the form of a speech signal or in the form of a text string. The user command 301 is pre-processed by the client application 303 to mask personal information as described herein. In this example, the pre-processed user command 305 (also referred to as the masked user command) is “Please copy the treatment notes used for XXXXX into YYYYY”. Here, ‘XXXXX’ represents a string of random characters that replaces the name ‘Jane Doe’. The name ‘Jane Doe’ may be identified as described herein. Similarly, ‘YYYYY’ represents a string of random characters that replaces the name ‘John Smith’. The names ‘Jane Doe’ and ‘John Smith’ may be identified as described herein. The names ‘Jane Doe’ and ‘John Smith’ may be stored in a list (such as list 2033 of FIG. 2) at the client application.

The pre-processed user command 305 is provided to the chatbot 307. The chatbot 307 identifies the one or more actions and/or one or more arguments 308 from the masked user command 305. Here, the identified action is to copy treatment notes (“copyNotes”) and the identified arguments are: the name of the source (“source_name”) “XXXXX”, and the name of the target (“target_name”) “YYYYY” The chatbot treats and processes the values “XXXXX” and “YYYYY” as if they are the original subjects of the user prompt. An example of a syntax for the action to copy treatment notes (“copyNotes”) is described in relation to FIG. 1.

The identified action and/or argument are then provided to the client application 309. The client application 309 then processes medical data with a feature of the client application, based on the identified action and/or argument. The client application 309 may also use the names ‘Jane Doe’ and ‘John Smith’ that have been stored to unmask (also referred to as de-anonymize) the action and/or argument. For example, the client application 309 may replace ‘XXXXX’ by ‘Jane Doe’ and ‘YYYYY’ by ‘John Smith’ in order to process medical data. Processed medical data may be output as described herein.

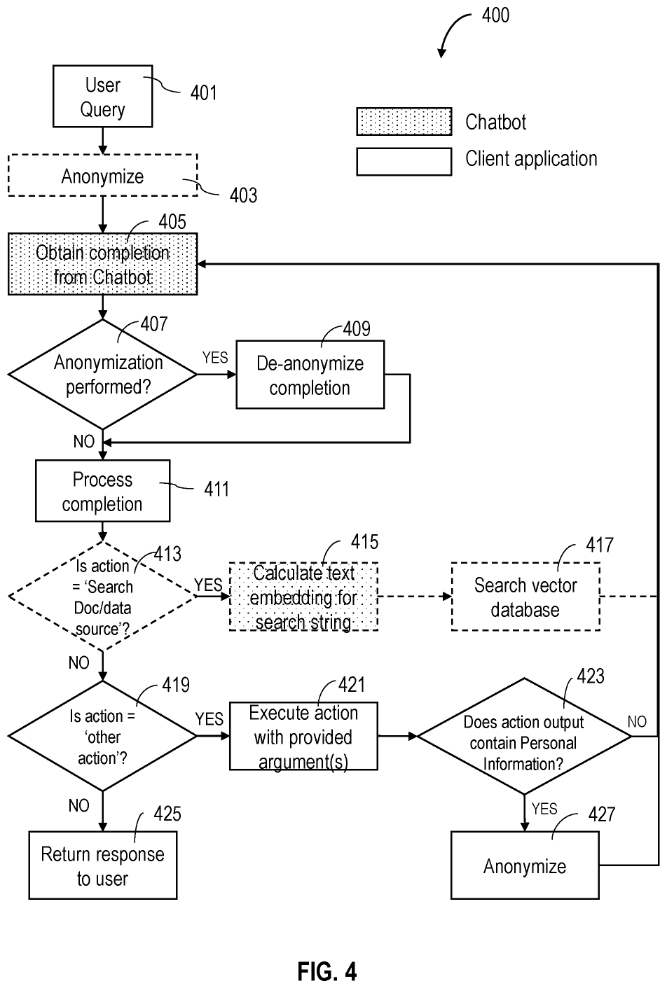

FIG. 4 shows a flowchart of a method 400 for processing medical data by a client application and a chatbot. The method 400 is for processing medical data associated with radiotherapy. The method of FIG. 4 may be performed by the system 600 described in relation to FIG. 6. In FIG. 4, operations performed by the chatbot are shown shaded, while operations performed by the client application are shown as blank boxes.

At operation 401 a user query is obtained. The user query 401 corresponds to the user command described herein.

At operation 403, the user query 401 is anonymized. Anonymization may correspond to pre-processing operation 103 described herein. Anonymization is performed when the user query comprises personal information. Anonymization may not be performed in cases where no personal information is detected. Thus, at operation 401 and 403, the user command is received, and responsive to determining that the user command comprises personal information, the user command is pre-processed such that personal information is anonymized (masked).

At operation 405, a completion is obtained from the chatbot. The chatbot is described in more detail in relation to FIG. 5. The chatbot may be invoked by providing the chatbot with a prompt that is based on the anonymized user query from operation 403. The prompt may be as described in relation to FIG. 1, FIG. 2, or FIG. 3. The completion may comprise one or more actions and/or one or more arguments. Alternatively, the prompt may be based on the output of operation 417, operation 423, or operation 427, which are described below.

At operation 407, it is checked, by the client application, whether the user query has been anonymized. For example, as described herein, anonymization comprises masking a term. The masked term may be stored in a list at the client application. By checking the list, it can be determined whether the anonymization had been performed.

If it is determined, at operation 407, that anonymization had been performed, the completion is de-anonymized at operation 409. De-anonymization corresponds to unmasking as described herein. The de-anonymized completion is then processed by the client application at operation 411. If it is determined, at operation 407, that anonymization had not been performed, the completion is processed, by the client application, at operation 411. Processing the completion at operation 411 corresponds to operation 107 of FIG. 1, for example.

At operation 413, it is checked by the client application whether the processed completion comprises an action to search a document or data source. For example, the document or data source relates to an instruction for use (IFU) document. For example, the IFU relates to the client application. If the action is to search a document or data source, operations 415 are 417 are performed. It is noted that operations 413, 415 and 417 are optional operations that are included when the use case comprises searching a document or a data source.

At operation 415, the chatbot calculates an embedding corresponding to the search string (which is an argument in the user query). The search string represents what to search for (i.e. a query or an item to be searched). The embedding may be understood as a vector representation of the search string. The search string is based on an argument from the processed completion. The embedding may be determined using a predefined embedding model (e.g., the text-embedding-ada-002 model) that converts a search string to an embedding.

At operation 417, the client application searches a vector database, the vector database corresponding to the document or data source. The vector database to be searched may be determined from an argument in the processed completion. Alternatively, the vector database to be searched is predefined. For example, when the determined action at operation 413 to search for information concerning the operation of the client application, the vector database corresponding to the IFU for the client application is searched. The vector database comprises one or more reference embeddings. Additionally and optionally, the vector database comprises a PostgreSQL database that stores reference embeddings. The reference embeddings may be obtained by using a predefined embedding model (e.g., the text-embedding-ada-002 model) to convert a text corpus (such as an IFU document) into said reference embeddings. At operation 417, the one or more reference embeddings are searched to find one or more relevant matches corresponding to the query. The one or more relevant matches may be referred to as candidate embeddings. Relevance may be computed using the cosine similarity between reference embeddings and the embedding representing the search string. The vector database may be accessed by the client application.

After operation 417, the method returns to operation 405. The outcome of operation 417 comprises one or more reference embeddings that are similar to the search string. Items from the data source (e.g. passages of text) that correspond to the reference embeddings may also be retrieved. A prompt comprising the retrieved items (e.g. passages of text), and the user query is then provided to the chatbot. At operation 405, the chatbot determines a completion based on said prompt.

For example, a user query (operation 401) may be: “how do I update a prescription in the client application”. No anonymization (operation 403) would be performed since the query does not include personal information. The query is passed to the chatbot at operation 405, where the action is determined to be to search the IFU document for the client application. The argument is determined to be ‘update a prescription’. The action and argument are passed to the client application for processing at operation 411. The action is determined to be to search a document (operation 413) and operations 415 and 417 are then executed. In more detail, an embedding corresponding to ‘update a prescription’ is obtained (operation 415), and relevant text passages from the IFU are retrieved at operation 417. The prompt to the chatbot may comprise instructions to generate a response in natural language based on the user query and the retrieved passages. The completion from the chatbot at operation 405 may be: “You may update a prescription by performing steps X, Y and Z, as described in section 00X of the documentation”. Since the completion does not contain any actions or personal information, operations 407, 413, and 419 would be skipped and the completion would be returned to the user at operation 425.

At operation 419, it is checked by the client application whether the processed completion (operation 411) comprises an action other than an action to search a document or data source. If so, operations 421 are 423 are performed. At operation 421, the action is executed, based on the provided arguments. Operation 421 corresponds to operation 107 of FIG. 1. At operation 423, it is checked whether the output of the executed action comprises personal information. If not, the method returns to operation 405. A prompt comprising the output of the executed action and, optionally, the user query, is provided to the chatbot (operation 405). If the output (e.g. processed medical data) does comprise personal information, the output is anonymized at operation 427, and a prompt comprising the anonymized output (e.g., masked medical data or masked processed medical data) is provided to the chatbot (operation 405). The prompt optionally includes the user query (anonymized if personal information is present). The anonymization operation 427 corresponds to operation 403. At operation 405, the chatbot determines a completion based on the received prompt. At operation 407 it is checked whether the completion has been anonymized. If so, the completion is de-anonymized at operation 409. The completion (from operation 405), or the de-anonymized completion (from operation 409), is then processed at operation 411. The processed completion may then be returned to the user at operation 425.

For example, a user query (operation 401) may correspond to the example of FIG. 3: “Please copy the treatment notes used for Jane Doe into John Smith”. Anonymization (operation 403) is performed to mask the names ‘Jane Doe’ and ‘John Smith’, as described in relation to FIG. 3. The query is passed to the chatbot at operation 405, where the action is determined to be to ‘copyNotes’ and the arguments are determined to be ‘source_name: XXXX’ and ‘target_name: YYYY’. At operation 407, it is determined that anonymization has been performed. The action and/or arguments are de-anonymized at operation 409, such that the action is ‘copyNotes’, while the arguments are: ‘source_name: Jane Doe’ and ‘target_name: ‘John Smith’. The de-anonymized action and arguments are processed by the client application at operation 411. The action is determined to be an action other than ‘search document/data source’ (operation 419) and operation 421 is performed. The outcome of operation 421 is that the patient notes for Jane Doe are copied into patient John Smith. The return value of the function may be “Notes copied from Jane Doe to John Smith”. The return value may be considered as an outcome of operation 421. The return value may be considered an outcome of the processing of medical data. The return value (“Notes copied from Jane Doe to John Smith”) is determined to contain personal information such as patient names (Jane Doe and John Smith) at operation 423. Operation 427 is then executed to anonymize the response. Anonymizing the response may be performed in a similar manner to operation 103 of FIG. 1. The prompt to the chatbot may comprise the anonymized user query and the anonymized response. The completion from the chatbot at operation 405 may be: “Treatment notes have been copied from XXXX to YYYY as requested”. The anonymized values XXXX and YYYY in the chatbot completion are replaced with “Jane Doe” and “John Smith” by the client application at operation 409. Since the processed completion (operation 411) does not contain any actions, operations 413, and 419 would be skipped and the completion would be returned to the user at operation 425.

At operation 425, the completion obtained from the chatbot (operation 405) is returned to the user.

FIG. 5 shows a schematic illustration of a language model 500 used in a chatbot. A language model is configured to predict and/or generate plausible words for a given input. The language model 500 comprises a neural network. The language model 500 is based on a Transformer 502. The transformer is a type of neural network that uses a self-attention mechanism. The transformer model is described in Vaswani et al. “Attention is all you need.” Advances in neural information processing systems 30 (2017), for example.

In particular, the language model 500 comprises a Generative Pre-trained Transformer (GPT) network 502. Language models comprising a GPT network may be referred to as Large Language Model (LLM).

Transformer models are able to handle long term dependencies in text and/or natural language processing and may be used in chatbots. The chatbot may comprise other components (not shown) such as components for preprocessing text before it is fed to the language model, or components for postprocessing the output of the language model before returning a response to the user. Preprocessing operations include operations such as tokenization (i.e. the splitting of a text string into smaller sequences of characters, referred to as tokens). The postprocessing operations depend on whether the language model is used for natural language inference, question answering tasks, similarity assessment tasks, classification tasks and so on.

The text and position embedding 501 represents the input to the transformer (or GPT) 502. The text and position embedding 501 corresponds to an encoded form of the user query described herein. To obtain the text and position embedding, bytepair encoding (BPE) may be used to covert the text string corresponding to the user query into a sequence of tokens. The tokens are selected from a predetermined vocabulary of tokens. The size of the vocabulary is denoted by V. For example, the vocabulary comprises between 32,000 and 64,000 tokens (i.e. V may be between 32,000 and 64,000). Each token is represented by a token embedding. The dimensionality, D, of the token embedding may be any of 768, 1024, 1280 or 1600, for example. The vocabulary may be represented by a token embedding matrix of size V×D. The token embedding matrix may be denoted Wₑ. Further, for each token in the sequence, a positional encoding vector (which indicates the order of the tokens in the sequence of tokens provided to the transformer), having the same dimension as the token embedding (D), is added to the token embedding. For example, the positional encoding vector may represent any one of 1024 positions in the input sequence. The positional encoding vector may be a learned (i.e. it comprises parameters that are determined during training). The positional encoding vector is denoted Wₚ. The length of the input sequence (e.g. 1024) may be referred to as the context size. The text and position embedding 501 is obtained based on the token embedding matrix and the positional encoding vector. The text and position embedding 501 comprises a sequence of vectors, representing the user query, where each vector has length D.

The language model 500 shown in FIG. 5 comprises a transformer network 502. The network 502 comprises a stack of twelve (12×) decoder blocks 503. Each decoder block 503 comprises a masked self-attention layer 505 and a feed forward neural network 507. The first decoder block receives a sequence of vectors, each vector representing a token, from the text and position embedding 501. Layer normalization (not shown) may be provided at the input of each masked self-attention layer 505 and at the input of each feed forward neural network 507. After the final (twelfth, in this case) self-attention layer, a further layer normalization may be included. The purpose of layer normalization is to provide smoother gradients, faster training and improved accuracy.

For ease of explanation, consider a sequence of four tokens, represented by four vectors x₁, x₂, x₃ and x₄. Each of x₁, x₂, x₃ and x₄ has a length D.

The tokens are fed into the masked self-attention layer 505 of the first decoder block 503. For each token, a query vector, q, a key vector, k, and a value vector, v, is obtained. The query, key, and value vectors for each token are obtained by multiplying weight matrices W^{Q}, W^{K} and W^{V} by the token vector. The weight matrices W^{Q}, W^{K} and W^{V} have a size of D. The weight matrices comprise weights that are determined during the training of the network. For each token i, the query vector qi is multiplied (dot product) by the key vectors (for all tokens) to get a score that indicates how well the other tokens match with the current token. In masked self-attention, the scores for future tokens (future tokens are those tokens that appear after the current token in the sequence) are set to 0. The value vector, vi, for each token are multiplied by their respective scores and then summed up to obtain a vector denoted as z. For x₁, x₂, x₃ and x₄, corresponding vectors z₁, z₂, z₃, and z₄ are obtained. z₁, z₂, z₃, and z₄ are the outputs of the masked self-attention layer 505. These are then presented to the feed forward neural network layer 507 of the decoder block. The feed forward NN is a fully-connected NN where the vector zᵢ for each token i is projected (by multiplying by further matrices-those matrices comprise weights that are determined during the training of the network) onto a result vector Rⱼ.

The result vectors for each decoder block 503 are passed on as an input to the next decoder block. Each block 503 comprises its own weight matrices (determined during model training). For the final decoder block, the result vectors are used to derive the prediction 509 of the transformer model. In more detail, each result vector is multiplied by the token embedding matrix. The result of this multiplication corresponds to a score for each of the V tokens in the vocabulary. This result may be taken as the prediction 509. In some examples, the token with the highest score is selected and used to form the prediction. In another example, the top scoring k tokens are considered. For example, k=40. The model iterates through all tokens until the end of the sequence is reached.

The language model 500 may be trained as follows. The training procedure may comprise two stages: (i) unsupervised pre-training, and (ii) supervised fine-tuning.

In the unsupervised pre-training stage, given an unsupervised corpus of tokens [symbol] ={u₁, . . . uₙ}, a standard language modelling objective is used to maximize a likelihood function. For example the likelihood function is:

$${L}_{1} ( 𝒰 ) = \sum log P ( {u}_{i} \vert {u}_{i - k} , … , {u}_{i - 1} : ⊖ ) \qquad EQUATION 1$$

Where k is the context size, and the conditional probability P is modelled using a neural network with parameters Θ. The corpus of tokens may be obtained from a dataset comprising ˜8 million documents (that corresponds to 40 GB of text). An example is the WebText corpus by OpenAI. Alternatively, the corpus of tokens may be obtained from a dataset such as the BooksCorpus. The parameters may be determined using stochastic gradient descent. For example, an Adam optimization scheme (with a maximum learning rate of 2.5e-4) may be used.

The language model 500 applies the following operation to the inputted token to produce an output distribution over target tokens:

$${R}_{o} = {UW}_{e} + {W}_{p} \qquad EQUATION 2$$

$${R}_{l} = transformer ( {R}_{l - 1} ) ∀ l ∈ [ 1 , n ]$$

$$P ( u ) = soft max ( {R}_{n} {W}_{e} )$$

Here, U is the vector of tokens, n represents the number of decoder blocks (n=12 in FIG. 5), R₀ represents the embedding 501 that is fed into the transformer 502, R_{I}=transformer (R_{I-1}) represents the operation applied by the Ith decoder block of the transformer to the result vector R_{I-1} from the (I−1)^{th} block, and P(u)=softmax(RₙWₑ) represents the application of the softmax function to the product of the result vector Rₙ from the last decoder block and the token embedding matrix Wₑ to provide an output distribution.

In the supervised fine-tuning stage, the pre-trained model from the first stage is further trained using a labelled dataset [symbol] . The dataset [symbol] comprises a sequence of input tokens c¹, . . . , c^{m} and a label y. The inputs are fed into the pre-trained model to obtain a result vector R^{m}ₙ from the last block of the transformer. The result vector is R^{m}ₙ is fed into a further linear output layer with parameters W_{y} to obtain a prediction for y as follows: P(y| c¹, . . . , c^{m})=softmax(R^{m}ₙW_{y}).

The objective to maximize in the supervised fine-tuning stage is:

$${L}_{2} ( 𝒞 ) = \sum log P ( y \vert {c}^{1} , … , {c}^{m} ) \qquad EQUATION 3$$

As for the first stage, the parameters may be determined using stochastic gradient descent. For example, an Adam optimization scheme (with a maximum learning rate of 2.5e-4) may be used.

The model described in FIG. 5 comprises a single attention head. Alternatively, the model may comprise multiple attention heads. For example, the model may comprise twelve attention heads.

Alternatively, the language model 502 comprises an architecture as described in Radford, A, et al. “Improving Language Understanding by Generative Pre-Training.” OpenAI.

Alternatively, the language model 502 comprises a GPT-2 architecture as described in Radford et al. “Language models are unsupervised multitask learners.” OpenAI blog 1.8 (2019): 9.

Alternatively, the language model 502 comprises a GPT-3 architecture as described in Brown, T. et al. “Language models are few-shot learners”. Advances in neural information processing systems, 33, 1877-1901 (2020).

Yet alternatively, the language model 502 comprises a transformer-based architecture as described in Touvron et al. “Llama 2: Open foundation and fine-tuned chat models.” arXiv preprint arXiv: 2307.09288 (2023).

Yet alternatively, the language model 502 comprises architectures based on GPT-4 or PaLM2.

Yet alternatively, the language model 502 comprises recurrent neural network (RNN) or a long short-term memory (LSTM) network.

FIG. 6 is a block diagram of an example of an apparatus, device, system or machine 600 upon which any one or more of the techniques (e.g., methods) discussed herein may perform. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act or operate as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The system 600 may receive a user command by way of any of input device 612, UI navigation device 614, display device 610 or microphone (optional) as described herein. The system 600 may output an indication that an action has been performed by way of any of display device 610 and signal generation device 618. The system 600 may output processed medical data in machine readable medium 622 as described herein. The system 600 may be used to implement the operations performed by the client application and/or the chatbot.

As described herein the chatbot may not be permitted to process and/or receive personal information associated with the medical data. On the other hand, the client application is configured to process medical data (e.g., it may be permitted to access and/or process medical data that comprises personal information). Thus, other than through the prompt and/or completion, the chatbot may not access medical data. In an example, the client application and the chatbot may be executed on separate machines. In another example, the client application and the chatbot may use different machine readable media 622. In another example, the client application and chatbot may use the machine 600 and medical data for the client application is isolated from the chatbot (such that the chatbot may not access the medical data). In yet another example, the client application and chatbot may use the machine readable medium 622 but medical data for the client application is isolated from the chatbot (such that the chatbot may not access the medical data).

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out or conduct a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out or conduct portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, field programmable gate array (FPGA), or any combination thereof), a main memory 604 and a static memory 606, some or all of which may communicate with each other via an interlink (e.g., bus) 630. The machine 600 may further include a display unit 610, an input device 612 (e.g., a keyboard or other alphanumeric input device), and a user interface (UI) navigation device 614 (e.g., a mouse). In an example, the display unit 610, input device 612 and UI navigation device 614 may be a touch screen display. Optionally, the machine 600 includes a microphone (e.g., to receive audio of verbal instructions that are converted to text input). The machine 600 may additionally include a storage device 608 (e.g., drive unit or other similar mass storage device or unit), a signal generation device 618 (e.g., a speaker), a network interface device 620 connected to a network 626, and one or more sensors 616, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 608 may include a machine readable medium 622 on which is stored one or more sets of data structures or instructions 624 (e.g., software) embodying or used by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 608 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

### Additional Notes and Examples

Unless specifically stated otherwise, as apparent from the following discussion, it is appreciated that throughout the description, discussions utilizing terms such as “receiving”, “determining”, “comparing”, “enabling”, “maintaining,” “identifying,”, “obtaining”, “accessing” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

While certain embodiments have been described, these embodiments have been presented by way of example only, and are not intended to limit the scope of the inventions. Indeed, the novel methods and apparatus described herein may be embodied in a variety of other forms; furthermore, various omissions, substitutions and changes in the form of methods and apparatus described herein may be made.

### Examples

Aspects and features of the present disclosure are set forth in the following numbered examples:

Example 1 is a method for processing medical data by a client application and a chatbot, the method comprising: receiving, by the client application, a user command comprising natural language, the user command further comprising personal information; pre-processing, by the client application, the user command to mask the personal information; invoking the chatbot to determine an action and/or an argument from the pre-processed user command; and processing the medical data, by the client application, using a feature of the client application to perform the chatbot-determined action and/or argument on the medical data.

Example 2 includes the method of Example 1 comprising: outputting an indication that the medical data has been processed; or outputting the processed medical data.

Example 3 includes the method of Example 2 wherein outputting an indication comprises: in response to determining, by the client application, that the processed medical data comprises personal information: masking, by the client application, the processed medical data; invoking the chatbot to generate an indication based on the masked medical data; and outputting, by the client application, the generated indication.

Example 4 includes the method of any preceding Example comprising: in response to determining, by the client application, that the user command comprises personal information: pre-processing, by the client application, the user command to mask the personal information.

Example 5 is the method of any preceding Example wherein the chatbot is configured to: determine an action from the pre-processed user command by comparing the pre-processed user command to a set of reference actions.

Example 6 is the method of Example 5 wherein the chatbot is configured to: determine an argument from the pre-processed user command by comparing the pre-processed user command to a set of reference arguments.

Example 7 is the method of any preceding Example wherein the chatbot-determined action comprises any one or more of: searching for patient records, querying patient records, creating a collection of patient records, modifying patient records, retrieving medical images, or searching a data source for an item.

Example 8 is the method of Example 7 comprising, in response to the determined action comprising searching a data source for an item: invoking the chatbot to determine an embedding that corresponds to the item to be searched; comparing, by the client application, the determined embedding with a set of reference embeddings; in response to the comparing (or another comparison), determining, by the client application, a candidate embedding; retrieving, by the client application, information corresponding to the candidate embedding from the data source.

Example 9 is the method of any of Example 1 to 8, wherein pre-processing, by the client application, the user command to mask personal information comprises removing the personal information from the user command.

Example 10 is the method of any of Examples 1 to 8, wherein pre-processing, by the client application, the user command to mask personal information comprises replacing the personal information by an anonymized text string.

Example 11 is the method of Examples 9 or 10 comprising storing the personal information at the client application.

Example 12 is the method of Example 11 comprising processing medical data, by the client application, using the stored personal information, and any of the determined action and/or the argument.

Example 13 is the method of Example 10 comprising: storing the personal information at the client application, and wherein processing medical data, by the client application, comprises replacing the anonymized text string with the stored personal information.

Example 14 is the method according to any preceding Example, wherein the chatbot comprises a language model.

Example 15 is the method according to Example 14, wherein the language model comprises a generative pre-trained transformer (GPT).

Example 16 is the method according to any preceding Example, wherein medical data comprises any one or more of electronic medical records, electronic health records, radiotherapy plans, patient data, and medical imaging data.

Example 17 is a system for processing medical data by a client application and a chatbot, the system comprising: processing circuitry; and memory, including instructions stored thereon, which, when executed by the processing circuitry, cause the processing circuitry to: perform the method of any of Examples 1 to 16.

Example 18 is a non-transitory computer-readable medium with instructions stored thereon that, when executed by a processor of a computing device, cause the processor to: perform the method of any of Examples 1 to 16.

Example 19 is a computer program comprising instructions which, when the program is executed by a computer, causes the computer to carry out the method of any of Examples 1 to 16.

What is claimed is:

1. A method for processing medical data by a client application and a chatbot, the method comprising:
receiving, by the client application, a user command comprising natural language, the user command further comprising personal information;
pre-processing, by the client application, the user command to mask the personal information;
invoking the chatbot to determine an action or an argument from the pre-processed user command; and
processing the medical data, by the client application, using a feature of the client application to perform the chatbot-determined action or argument on the medical data.

2. The method of claim 1, comprising:
outputting an indication that the medical data has been processed; or
outputting the processed medical data.

3. The method of claim 2, wherein outputting an indication comprises:
in response to determining, by the client application, that the processed medical data comprises personal information:
masking, by the client application, the processed medical data;
invoking the chatbot to generate an indication based on the masked medical data; and
outputting, by the client application, the generated indication.

4. The method of claim 1, comprising:
in response to determining, by the client application, that the user command comprises personal information:
pre-processing, by the client application, the user command to mask the personal information.

5. The method of claim 1, wherein the chatbot is configured to:
determine an action from the pre-processed user command by comparing the pre-processed user command to a set of reference actions.

6. The method of claim 5, wherein the chatbot is configured to:
determine an argument from the pre-processed user command by comparing the pre-processed user command to a set of reference arguments.

7. The method of claim 1, wherein the chatbot-determined action comprises any one or more of: searching for patient records, querying patient records, creating a collection of patient records, modifying patient records, retrieving medical images, or searching a data source for an item.

8. The method of claim 7, comprising, in response to the determined action comprising searching a data source:
invoking the chatbot to determine an embedding that corresponds to the item to be searched;
comparing, by the client application, the determined embedding with a set of reference embeddings;
in response to the comparing, determining, by the client application, a candidate embedding; and
retrieving, by the client application, information corresponding to the candidate embedding from the data source.

9. The method of claim 1, wherein pre-processing, by the client application, the user command to mask personal information comprises removing the personal information from the user command.

10. The method of claim 1, wherein pre-processing, by the client application, the user command to mask personal information comprises replacing the personal information by an anonymized text string.

11. The method of claim 9, comprising storing the personal information at the client application.

12. The method of claim 11, comprising processing medical data, by the client application, using the stored personal information, and the determined action or the argument.

13. The method of claim 10, comprising:
storing the personal information at the client application, and
wherein processing medical data, by the client application, comprises replacing the anonymized text string with the stored personal information.

14. The method of claim 1, wherein the chatbot comprises a language model.

15. The method of claim 14, wherein the language model comprises a generative pre-trained transformer (GPT).

16. The method of claim 1, wherein medical data comprises any one or more of electronic medical records, electronic health records, treatment plans, patient data, and medical imaging data.

17. A system for processing medical data by a client application and a chatbot, the system comprising:
processing circuitry; and
memory, including instructions stored thereon, which, when executed by the processing circuitry, cause the processing circuitry to:
receive, by the client application, a user command comprising natural language, the user command further comprising personal information;
pre-process, by the client application, the user command to mask the personal information;
invoking the chatbot to determine an action or an argument from the pre-processed user command; and
process the medical data, by the client application, using a feature of the client application to perform the chatbot-determined action or argument on the medical data.

18. A non-transitory computer-readable medium with instructions stored thereon that, when executed by a processor of a computing device, cause the processor to:
receive, by a client application, a user command comprising natural language, the user command further comprising personal information;
pre-process, by the client application, the user command to mask the personal information;
invoking a chatbot to determine an action or an argument from the pre-processed user command; and
process the medical data, by the client application, using a feature of the client application to perform the chatbot-determined action or argument on the medical data.
